(19) United States
(12) Patent Application Publication
(10) Pub. No.: US 2008/0114453 A1
(43) Pub. Date: May 15, 2008

(54) INTERVERTEBRAL PROSTHETIC DEVICES AND SURGICAL METHODS

(75) Inventor: Thomas J. FRANCIS, Cordova, TN (US)

Correspondence Address: HESLIN ROTHENBERG FARLEY & MESITI P.C., 5 COLUMBIA CIRCLE, ALBANY, NY, 12203, US

(73) Assignee: WARSAW ORTHOPEDIC, INC., Warsaw, IN (US)

(21) Appl. No.: 11/559,035

(22) Filed: Nov. 13, 2006

(51) Int. Cl. A61F 2/44

(52) U.S. Cl. 623/17.14; 623/17.15

(57) ABSTRACT

Intervertebral prosthetic devices and methods of implanting the same within an intervertebral space between adjacent vertebral bodies are provided. One device includes first and second components, each configured to engage a respective one of the adjacent vertebral bodies. Each component includes a vertebral support plate with a spherical segment protruding therefrom. The spherical segments are disposed to reside within nuclear recesses in the adjacent vertebral bodies when the device is implanted in the intervertebral space, and are configured with a common center of rotation which aligns with the natural axis of rotation of the adjacent vertebral bodies. An articulation member, which interfaces the first and second vertebral support plates, may be an articular ball and respective concave articular surface regions in the plates, or alternatively, a convex articular surface and a respective concave articular surface in the vertebral support plates of the first and second components.

### CROSS-REFERENCE TO RELATED APPLICATIONS

This application contains subject matter which is related to the subject matter of the following applications, which are hereby incorporated herein by reference in their entirety:

“Intervertebral Prosthetic Devices and Methods”, Eric Steven Heinz, U.S. Ser. No. 11/398,874, filed Apr. 6, 2006;

“Intervertebral Spinal Implant Devices and Methods of Use”, Heinz et al., U.S. Ser. No. 11/343,954 filed Jan. 31, 2006;

“Intervertebral Prosthetic Disc”, Heinz et al., U.S. Ser. No. 11/343,935, filed Jan. 31, 2006;

“Articular Disc Prosthesis and Method for Implanting the Same”, Eisermann et al., U.S. Ser. No. 10/412,700, filed Apr. 11, 2003, and published on Oct. 30, 2003 as Patent Application Publication No. US 2003/0204261 A1;

“Intervertebral Prosthetic Joint”, Eisermann et al., U.S. Ser. No. 10/620,529, filed Jul. 16, 2003, and published on Apr. 15, 2004 as Patent Application Publication No. US 2004/0073312 A1; and

“Artificial Intervertebral Disc for Lateral Insertion”, M. Peterman, U.S. Ser. No. 10/839,100, filed May 5, 2004, and published on Nov. 10, 2005 as Patent Application Publication No. US 2005/0251261 A1.

### TECHNCIAL FIELD

The present invention relates generally to spinal implants and methods, and more particularly, to intervertebral prosthetic joint devices and methods for use in total or partial replacement of a natural intervertebral disc.

### BACKGROUND OF THE INVENTION

In the treatment of disease, injuries and malformations affecting spinal motion segments, and especially those affecting disc tissue, it has been known to remove some or all of a degenerated, ruptured or otherwise failing disc. In cases involving intervertebral disc tissue that has been removed, or is otherwise absent from a spinal motion segment, corrective measures are typically desirable.

In one approach, adjacent vertebrae are fused together using transplanted bone tissue, an artificial fusion component, or other compositions or devices. Spinal fusion procedures, however, have raised concerns in the medical community that the biomechanical rigidity of the intervertebral fusion may predispose neighboring spinal motion segments to rapid deterioration. Unlike a natural intervertebral disc, spinal fusion prevents the fused vertebrae from pivoting and rotating with respect to one another. Such lack of mobility tends to increase stress on adjacent spinal motion segments. Additionally, conditions may develop within adjacent spinal motion segments, including disc degeneration, disc herniation, instability, spinal stenosis, spondylosis and facet joint arthritis as a result of the spinal fusion. Consequently, many patients may require additional disc removal and/or another type of surgical procedure as a result of the spinal fusion. Alternatives to spinal fusion are therefore desirable.

Alternative approaches to bone grafting employ a manufactured implant made of a synthetic material that is biologically compatible with a body in the vertebrae. There have been extensive attempts at developing acceptable prosthetic implants that can be used to replace an intervertebral disc and yet maintain the stability and range of motion of the intervertebral disc space between adjacent vertebrae. While many types of prosthetic devices have been proposed, there remains a need in the art for further enhanced intervertebral prosthetic disc devices and methods of implanting thereof.

### SUMMARY OF THE INVENTION

The shortcomings of the prior art are overcome and additional advantages are provided, in one aspect, through provision of an intervertebral prosthetic device which includes a first component and a second component. The first component is configured to engage a first vertebral body, and includes a first vertebral support plate having a first spherical segment protruding from a first surface thereof. The first spherical segment is disposed to reside within a nuclear recess in the first vertebral body when the intervertebral prosthetic device is implanted between the first vertebral body and a second vertebral body. The second component is configured to engage the second vertebral body, and includes a second vertebral support plate having a second spherical segment protruding from a first surface thereof disposed to reside within a nuclear recess in the second vertebral body when the intervertebral prosthetic device is implanted between the first and second vertebral bodies. An articulation member interfaces the first vertebral support plate and the second vertebral support plate, and the first spherical segment protruding from the first vertebral support plate and the second spherical segment protruding from the second vertebral support plate have a common center of rotation when the intervertebral prosthetic device is implanted between the adjacent vertebral bodies.

In another aspect, an intervertebral prosthetic device is provided which includes a first component and a second component. The first component is configured to engage a first vertebral body, and includes a first vertebral support plate having a first convex protrusion extending from a first surface and a convex articular protrusion extending form a second surface thereof. The first convex protrusion is disposed to reside within a nuclear recess in the first vertebral body when the intervertebral prosthetic device is implanted between the first vertebral body and a second vertebral body. The second component is configured to engage the second vertebral body, and includes a second vertebral support plate having a second convex protrusion extending from a first surface thereof disposed to reside within a nuclear recess in the second vertebral body when the intervertebral prosthetic device is implanted between the first and second vertebral bodies, and a concave articular recess in a second surface thereof configured and disposed to at least partially receive the convex articular protrusion extending from the second surface of the first vertebral support plate when the intervertebral prosthetic device is implanted between the first and second vertebral bodies. The convex articular protrusion and the concave articular recess function as an articulation member providing articulating motion for the intervertebral prosthetic device.

In a further aspect, a surgical method is provided which includes: creating a window in one of an anterior, lateral or posterior approach to an intervertebral disc space between adjacent first and second vertebral bodies; obtaining a motion-preserving prosthetic device comprising first and second components for implant, the first component being configured to engage the first vertebral body, and comprising a first vertebral support plate having a first spherical segment protruding from a first surface thereof, the first spherical segment being disposed to reside within a nuclear recess in the first vertebral body when the prosthetic device is implanted between the first and second vertebral bodies, and the second component being configured to engage the second vertebral body, the second component including a second vertebral support plate having a second spherical segment protruding from a surface thereof disposed to reside within a nuclear recess in the second vertebral body when the prosthetic device is implanted between the adjacent first and second vertebral bodies, and an articulation member interfacing the first vertebral support plate and the second vertebral support plate, wherein the first spherical segment protruding from the first vertebral support plate and the second spherical segment protruding from the second vertebral support plate share a common center of rotation; and inserting the motion-preserving prosthetic device through the window and into the intervertebral space with the first spherical segment protruding from the first vertebral support plate residing within the nuclear recess in the first vertebral body and the second spherical segment protruding from the second vertebral support plate residing within the nuclear recess in the second vertebral body.

In a yet further aspect, a surgical method is provided which includes: creating a window in one of an anterior, lateral or posterior approach to an intervertebral disc space between adjacent first and second vertebral bodies; obtaining a motion-preserving prosthetic device including first and second components, the first component being configured to engage the first vertebral body, and including a first vertebral support plate having a first convex protrusion extending from a first surface, and a convex articular protrusion extending from a second surface thereof, the first convex protrusion being disposed to reside within a nuclear recess in the first vertebral body when the intervertebral prosthetic device is implanted between the first and second vertebral bodies, and the second component being configured to engage the second vertebral body, the second component including a second vertebral support plate having a second convex protrusion extending from a first surface thereof disposed to reside within a nuclear recess in the second vertebral body when the prosthetic device is implanted between the first and second vertebral bodies, and having a concave articular recess in a second surface thereof configured and disposed to at least partially receive the convex articular protrusion extending from the second surface of the first support plate when the prosthetic device is implanted between the first and second vertebral bodies, wherein the convex articular protrusion and the concave articular recess function as an articulation member providing articulating motion for the prosthetic device; and inserting the motion-preserving prosthetic device through the window and into the intervertebral disc space with the first convex protrusion extending from the first vertebral support plate residing within the nuclear recess in the first vertebral body, and the second convex protrusion extending from the second vertebral support plate residing within the nuclear recess in the second vertebral body.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 is a lateral view of a portion of a spine, including two vertebral bodies and a disc disposed therebetweeen;

FIG. 2 is an isometric view of one embodiment of an intervertebral prosthetic device, in accordance with an aspect of the present invention;

FIG. 3 is an exploded view of the intervertebral prosthetic device of FIG. 2, in accordance with an aspect of the present invention;

FIG. 4 is an anterior view of the intervertebral prosthetic device of FIGS. 2 & 3, in accordance with an aspect of the present invention;

FIG. 5 is a lateral view of the intervertebral prosthetic device of FIGS. 2-4, in accordance with an aspect of the present invention;

FIG. 6 is a lateral view of a portion of a spine, showing two adjacent vertebral bodies with an intervertebral prosthetic device such as depicted in FIGS. 2-5, disposed therebetween, in accordance with an aspect of the present invention;

FIG. 7 is an isometric view of an alternate embodiment of an intervertebral prosthetic device, in accordance with an aspect of the present invention;

FIG. 8 is an isometric view of a further alternate embodiment of an intervertebral prosthetic device, in accordance with an aspect of the present invention;

FIG. 9 is an exploded view of the intervertebral prosthetic device of FIG. 8, in accordance with an aspect of the present invention;

FIG. 10 is an anterior view of the intervertebral prosthetic device of FIGS. 8 & 9, in accordance with an aspect of the present invention;

FIG. 11 is a lateral view of the intervertebral prosthetic device of FIGS. 8-10, in accordance with an aspect of the present invention;

FIG. 12 is an isometric view of another embodiment of an intervertebral prosthetic device, in accordance with an aspect of the present invention;

FIG. 13 is an isometric view of yet another embodiment of an intervertebral prosthetic device, in accordance with an aspect of the present invention;

FIG. 14 is an exploded view of the intervertebral prosthetic device of FIG. 13, in accordance with an aspect of the present invention; and

FIG. 15 is an elevational view of one embodiment of a trial instrument to be employed in sizing intervertebral space between nuclear recesses of adjacent vertebral bodies pursuant to a surgical method for implanting an intervertebral prosthetic device, in accordance with an aspect of the present invention.

### BEST MODE FOR CARRYING OUT THE INVENTION

This disclosure relates to intervertebral disc prostheses for anterior, lateral or posterior insertion, either direct or indirect. For purposes of promoting and understanding the principles of this disclosure, reference is made hereinbelow to the anterior insertion embodiments, or examples, illustrated in the drawings and specific language is used to describe the same. It will nevertheless be understood that no limitation of the scope of the disclosure is thereby intended. Any alterations and further modifications to the described embodiments, and any further applications of the principles of disclosure as described herein are contemplated as would normally occur to one skilled in the art to which this disclosure relates. As such, individual features of separately described embodiments can be combined to form additional embodiments.

Provided herein are various intervertebral prosthetic devices (i.e., articular disc prostheses) and methods of implanting the same. In the various embodiments presented, the intervertebral prosthetic device is a multi-component device, which includes first and second vertebral support plates having first and second convex protrusions, respectively, extending from vertebral bearing surfaces thereof. The first and second convex protrusions are configured and disposed to reside within respective nuclear recesses in the adjacent superior and inferior vertebral bodies when the device is inserted within an intervertebral disc space. As used herein, the phrase “nuclear recess” refers to a roughly spherical-shaped recess segment in the respective endplates of the adjacent vertebral bodies. These nuclear recesses (i.e., indents, divots, depressions, etc.) are often naturally occurring and are disposed somewhat posterior the vertebral body where the natural axis of rotation exists. More particularly, nuclear recesses typically form in the vertebral body endplates around the disc nucleus in patients with disc degeneration. With disc degeneration, the nucleus often becomes hardened due to dehydration, and the resultant loading on the disc causes nuclear recesses to be formed in the adjacent vertebral bodies along the natural axis of rotation. The shape of the nuclear recesses can vary as the degenerated nucleus is pushed into the endplates.

As an enhancement, nuclear recesses may be artificially formed via an appropriate surgical procedure. More particularly, the intervertebral space may be enlarged by forming or enhancing spherical recesses (i.e., nuclear recesses) along the superior and inferior portions of the upper and lower vertebral bodies. These spherical recess portions have a shape and configuration that substantially corresponds to the outer profile of the first and second convex protrusions extending from the vertebral bearing surfaces of the first and second vertebral support plates. In one embodiment, the nuclear recesses have an approximately hemi-spherical shape; however, other shapes and configurations of nuclear recesses are also contemplated, as would occur to one of skill in the art, including, for example hemi-cylindrical shapes.

An articulation member interfaces the first vertebral support plate and the second vertebral support plate. In certain implementations, the first and second convex protrusions are first and second spherical segments sized and disposed with a common center of rotation when the intervertebral prosthetic device is inserted within an intervertebral space between adjacent superior and inferior vertebral bodies. By disposing these spherical segments (protruding from the vertebral bearing surfaces of the first and second vertebral support plates) within the nuclear recesses of the adjacent vertebral bodies, the common center of rotation of the spherical segments aligns with the natural axis of rotation of the adjacent vertebrae.

In further implementations, the articulation member includes an articular ball, and first and second concave articular surface regions in opposing surfaces of the first and second vertebral support plates. In certain implementations, the first and second spherical segments (protruding from the vertebral bearing surfaces of the first and second vertebral support plates) and the articular ball are all sized and aligned to share a common center of rotation, which as in the initial embodiment, is aligned with the natural axis of rotation of the adjacent vertebrae through placement of the first and second spherical segments within the nuclear recesses of the adjacent vertebral bodies. In certain other implementations, the articulation member is a convex articular protrusion and a concave articular recess, formed in respective opposing surfaces of the first and second vertebral support plates, which together provide articulating motion for the intervertebral prosthetic device.

In additional embodiments, the first and second convex protrusions respectively comprise first and second spherical segments of common radius R, and the convex articular protrusion is an articular spherical segment and the concave articular recess is an articular spherical recess segment in the respective opposing surfaces of the first and second vertebral support plates. Within this configuration, the first and second spherical segments protruding from the vertebral bearing surfaces of the first and second vertebral support plates, as well as the articulating spherical segment and spherical recess segment are aligned along a central axis, and the central axis coincides with the natural axis of rotation between the adjacent vertebrae when the first and second spherical segments are disposed with the nuclear recesses thereof. With this configuration, the first and second spherical segments protruding from the first and second vertebral support plates, as well as the articulating spherical segment have a common center of rotation along the natural axis of rotation of the adjacent vertebrae.

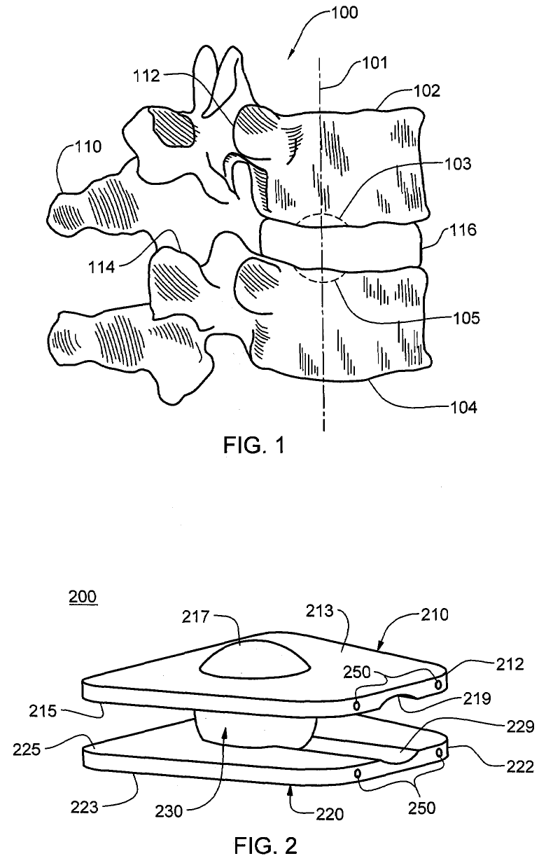

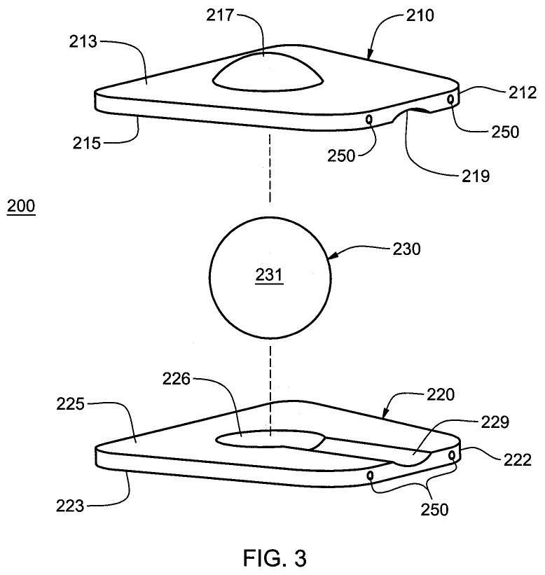

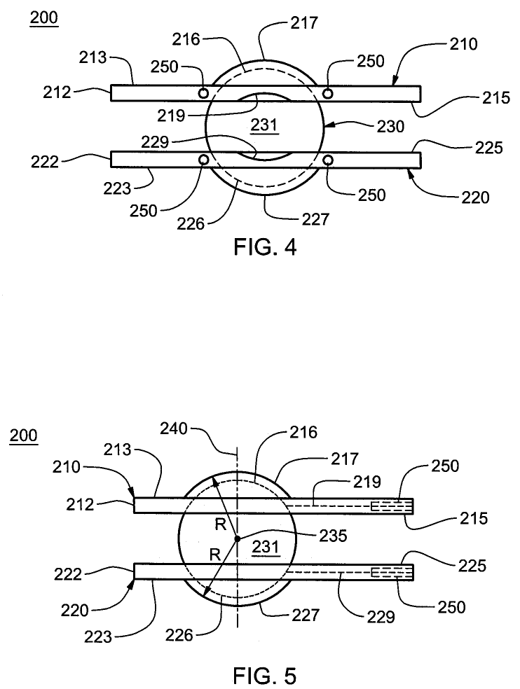

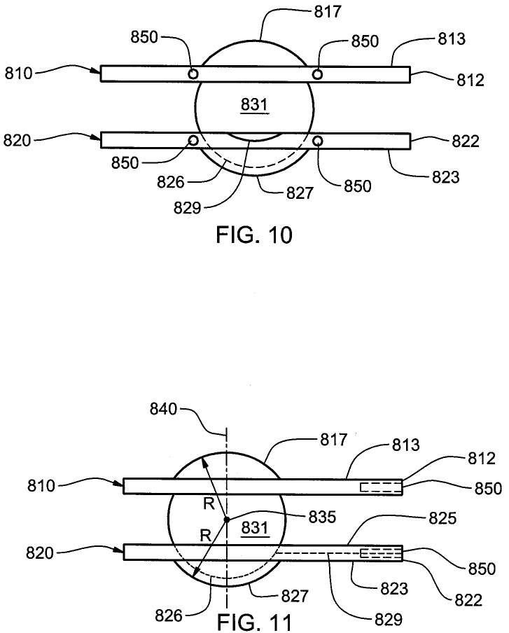

The above-outlined aspects of the present invention, as well as further aspects thereof, are described in greater detail below with reference to the embodiments illustrated in FIGS. 1-15. It should be noted again, however, that the various embodiments of FIGS. 1-15 are provided by way of example only, and that no limitation on the scope of the invention is intended by the embodiments depicted and described herein.

FIG. 1 illustrates a lateral view of a portion of a spine, generally denoted 100, comprising two vertebrae or vertebral bodies 102, 104, with a disc 116 shown therebetween. Each vertebral body 102, 104 comprises a generally cylindrical body that contributes to the primary weight-bearing portion of spine 100. As shown, each vertebral body 102, 104 further includes various boney processes 110, 112 extending posterior to the body. Adjacent vertebral bodies, 102, 104 move relative to each other via facet joints 114, and due to the flexibility of disc 116.

Each vertebral body 102, 104 comprises an outer cortical rim composed of cortical bone, with an inner cancellous bone disposed within the cortical rim. The cortical rim is often referred to as the apophyseal rim or apophyseal ring. Further, the cancellous bone is softer than the cortical bone of the cortical rim. Each vertebral body 102, 104 further comprises an endplate (not shown) composed of an outer layer of cartilage and an inner layer of bone that is strongly attached to the cortical rim of the vertebral body. A nuclear recess 103, 105 is naturally occurring within respective opposing endplates of the adjacent vertebral bodies along the natural axis of rotation 101 of spine 100 (e.g., due to disc degeneration). Size of the nuclear recesses can vary from patient to patient, as well as from vertebrae to vertebrae within a given patient. Thus, artificial enhancement via an appropriate surgical procedure of the nuclear recess may be desirable. Note that the nuclear recess employed herein is in addition to the general concavity of the opposing endplates and is in one example an approximately spherical-shaped recess segment naturally occurring along the axis of rotation of the adjacent vertebrae.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., the structures described above in conjunction with FIG. 1.

If intervertebral disc 116 is diseased, degenerated, damaged, or otherwise in need of replacement, the disc can be at least partially removed and replaced with an intervertebral prosthetic disc, such as illustrated in FIG. 2-14. The intervertebral disc can be removed via a discectomy, or similar surgical procedure, well known in the art. Removal of intervertebral disc material results in the formation of an intervertebral space (not shown) between the two adjacent vertebral bodies.

FIGS. 2-5 depict one embodiment of an intervertebral prosthetic device 200, in accordance with an aspect of the present invention. By way of example only, device 200 is shown as a multi-component 210, 220, 230 implant (i.e., a three-piece device). In this example, component 210 is a first vertebral support plate 212, component 220 a second vertebral support plate 222, and component 230 an articular ball 231. Vertebral support plates 212, 214 each include a first surface 213, 223 (i.e., a vertebral bearing surface) having a spherical segment 217, 227 protruding therefrom. Spherical segments 217, 227 are disposed somewhat posterior the vertebral body when implanted within the intervertebral space, and are positioned to align with the respective nuclear recesses in the adjacent vertebral bodies, and thus, align with the natural axis of rotation of the vertebrae. As shown in FIG. 5, the outer surfaces of spherical segments 217, 227 are disposed at radius R from a center point 235, meaning that spherical segments 217, 227 share a common center of rotation. The common center of rotation is also shared by articular ball 231, which as shown in FIG. 5, resides in respective spherical recess segments 216, 226 in second surfaces 215, 225 of vertebral support plates 212, 222 (i.e., in main opposing surfaces of the vertebral support plates). Note that the radius of the respective spherical recess segments 216, 226 may be slightly larger than the radius of articular ball 231 to facilitate articulating motion between the articular ball and the spherical recess segments 216, 226.

As noted, the first surfaces 213, 223 of vertebral support plates 212, 222 are configured as vertebral bearing surfaces and are sized to engage the apophyseal ring of the adjacent vertebral bodies when inserted within an intervertebral disc space. In the implementation illustrated, the vertebral support surfaces (i.e., first surfaces 213, 223) are shown free of any keel, anchor, spike, etc., designed to affix the implant relative to the adjacent vertebrae. This facilitates positioning of the device within the intervertebral space of a patient and then the ready adjusting of the device within the space as desired. In addition to a placement advantage, keel-less or anchor-less vertebral support surfaces may be beneficial with surgical techniques requiring an inter-operative flexion/extension radiograph, or other simulation of the implant's range of motion in situ. The device position could be adjusted until the prosthetic device is located within the anatomical center of rotation for the surrounding anatomy.

The vertebral support surfaces (i.e., first surfaces 213, 223) of vertebral support plates 212, 222 are designed to be in direct physical contact with the respective vertebral bodies and may be coated or textured to promote osteointegration. For example, a bone-growth promoting substance such as, for example, hydroxyapatite coating formed of calcium phosphate may be employed. Additionally, the first surfaces 213, 223, may be roughened prior to being coated with the bone-growth promoting substance to further enhance bone on-growth. Such surface roughening may be accomplished by way of, for example, acid etching, knurling, application of a bead coating (e.g., cobalt chrome beads), application of a roughening spray (e.g., titanium plasma spray (TPS)), laser blasting, or other methods of roughening that are known to one skilled in the art.

The articulating joint of intervertebral prosthetic device 200 depicted in FIGS. 2-5 provides relative pivotal and rotational movement between the adjacent vertebral bodies to maintain or restore motion substantially similar to the normal bio-mechanical motion provided by a natural intervertebral disc. Specifically, the articulating vertebral support plates 212, 222 are permitted to pivot relative to one another about a number of axes, including a lateral or side-to-side pivotal movement about a longitudinal axis, and an anterior-posterior pivotal movement about a transverse axis. Further, it should be understood that these articular components are permitted to pivot relative to one another about any axis that lies in a plane that intersects the longitudinal axis and the transverse axis. Additionally, the articular components are permitted to rotate relative to one another about a rotational axis. Although the articulating joint is illustrated and described as providing specific articulating motion, it should be understood that other combinations of articulating movement are also possible and are contemplated as falling within the scope of the present invention. Further, it should be understood that other types of arthroplasty implants allowing articulating movement are also contemplated, including for example, single-component and two-component prosthetic discs.

Although the components 210, 220, 230 of intervertebral prosthetic device 200 may be formed from a wide variety of materials, including metal-containing materials, polymer materials, or composite materials that include metals, polymers, or combinations of metals and polymers, in one embodiment, one or more of the components are formed of a cobalt-chrome-molybdenum metallic alloy (ASTM F-999 or F-75). However, in alternative embodiments, the support plates and/or articular ball may be formed of other materials, including ceramic material, other metallic material, such as titanium or stainless steel, polymeric material (such as polyurethane material, polyolefin material, polyether material, silicone material, or a combination thereof), or any other biocompatible material that would be apparent to one of ordinary skill in the art. Further, depending upon the implant configuration, articular ball 231 may comprise the same material as vertebral support plates 212, 222, or a different material. For example, nucleus 231 could be an implantable grade PEEK material. One example of a suitable medical grade material is marketed as PEEK® Optima available from Invibio, Inc., of Greenville, S.C., USA.

In the embodiment of FIGS. 2-5, intervertebral prosthetic device 200 further includes aligned troughs 219, 229, formed in second surfaces 215, 225 of vertebral support plates 212, 222, respectively. Troughs 219, 229 extend from an anterior edge of intervertebral prosthetic device 200 to spherical recess segments 216, 226, respectively, and are sized to facilitate insertion of articular ball 231 between the vertebral support plates after the vertebral support plates have been inserted into the intervertebral disc space in engagement with a respective one of the adjacent vertebral bodies. Sizing and placement of troughs 219, 229 can vary as desired for a particular intervertebral prosthetic device and a particular surgical implant method to be employed. Further, although shown for an anterior approach, troughs 219, 229 could alternately be disposed to extend laterally or posteriorly from spherical recess segments 216, 226 to a respective edge of the device, for example, for a lateral or posterior insertion approach, respectively.

Note that for a lateral or posterior insertion approach, the intervertebral prosthetic device depicted in FIGS. 2-5 could be redesigned, with the vertebral support plates being rectangular-shaped with rounded edges to facilitate insertion of the prosthetic device.

Referring further to FIGS. 2-5, the trough disposition illustrated assumes that the intervertebral prosthetic device is implanted through an anterior approach to the intervertebral disc space. The anterior approach may be either a direct anterior insertion trajectory or an oblique anterior insertion trajectory. If an oblique anterior insertion trajectory is employed, then troughs 219, 229 could be disposed to extend from an oblique anterior edge of the intervertebral prosthetic device to the spherical recess segments.

Insertion of the device, and more particularly, of vertebral support plates 212, 222 is facilitated by providing appropriately sized instrument-receiving notches 250 (or holes, bores, etc.) in an anterior edge of the device to allow for gripping of the vertebral support plates to aid in the manipulation and insertion of the plates in engaging position with the respective vertebral bodies within the intervertebral disc space.

Once implanted, the intervertebral prosthetic device undergoes compressive loading which will serve to maintain articular ball 231 within the spherical recess segments 216, 226 of the vertebral support plates 212, 222. Vertebral support plates 212, 222, being configured to engage the respective apophyseal rings of the adjacent vertebrae, are trapezoidal-shaped (in one embodiment) and designed to prevent subsidence of the articular member of the device, as might occur with prior ball-bearing-type devices.

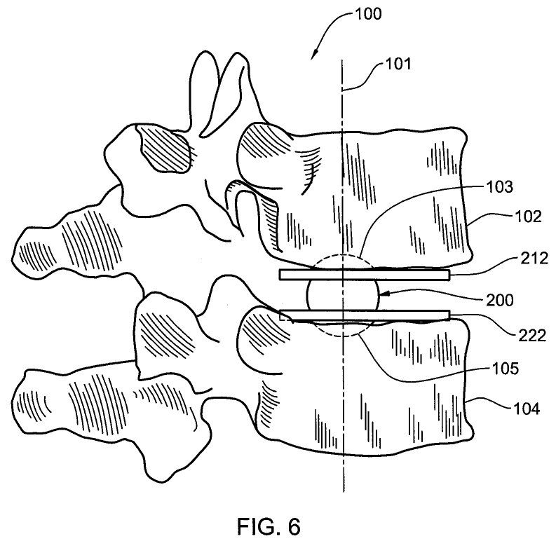

FIG. 6 illustrates a lateral view of a portion of spine such as depicted in FIG. 1, with the intervertebral disc thereof having been removed and an intervertebral prosthetic device 200, such as depicted in FIGS. 2-5, inserted in operative position between the adjacent vertebral bodies 102, 104. In operative position, the spherical segments protruding from the vertebral support plates of the prosthetic device 200 are disposed within the nuclear recesses 103, 105 of vertebral bodies 102, 104, and thus, align the center of rotation of the intervertebral prosthetic device with the natural axis of rotation 101 of spine 100.

Various enhancements to the intervertebral prosthetic device of FIGS. 2-5 are possible. For example, although spherical recess segments 216, 226 are illustrated as having a generally smooth, uninterrupted articular surface, it should be understood that a surface depression or cavity may be defined along a portion of these recesses to provide, for example, a means for clearing out matter, such as particulate debris, that is disposed between the abutting articular components.

In certain embodiments, vertebral support plates 212, 222 are identical in shape, while in other embodiments, the vertebral support plates may be of different sizes and shapes to accommodate different requirements. For example, in alternate implementations, the radius of curvature of the spherical segments 217, 227 may vary, for example, to accommodate different sized nuclear recesses in the adjacent vertebral bodies.

In other implementations, a combination of holes, apertures, and other mechanisms can be used to engage with various surgical instruments to allow the manipulation and insertion of the components of the intervertebral prosthetic device into an intervertebral disc space between adjacent vertebral bodies.

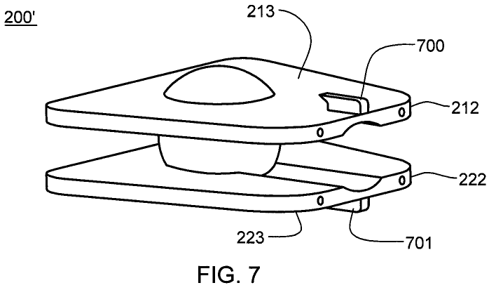

FIG. 7 depicts a further enhancement to an intervertebral prosthetic device 200′, in accordance with an aspect of the present invention. In this implementation, intervertebral prosthetic device 200′ is substantially identical to intervertebral prosthetic device 200 of FIGS. 2-5, except for the addition of gripping elements 700, 701 to the first surfaces 213, 223 of the vertebral support plates 212, 222. Gripping elements 700, 701 are generally any element for gripping vertebral bone (such as keels, spikes, flanges, etc.) and are added to the vertebral support plates 212, 222 to facilitate securing the plates to the respective vertebral bodies. It should be understood that the number and location of gripping elements 700, 701 may vary. Depending on the implementation, gripping elements 700, 701 may include openings (not shown) extending therethrough to facilitate bone-through growth to enhance fixation to the adjacent vertebrae. The surfaces of gripping elements 700, 701 may further be coated with a bone-growth material, such as hydroxyapatite, to promote bony engagement with the adjacent vertebrae.

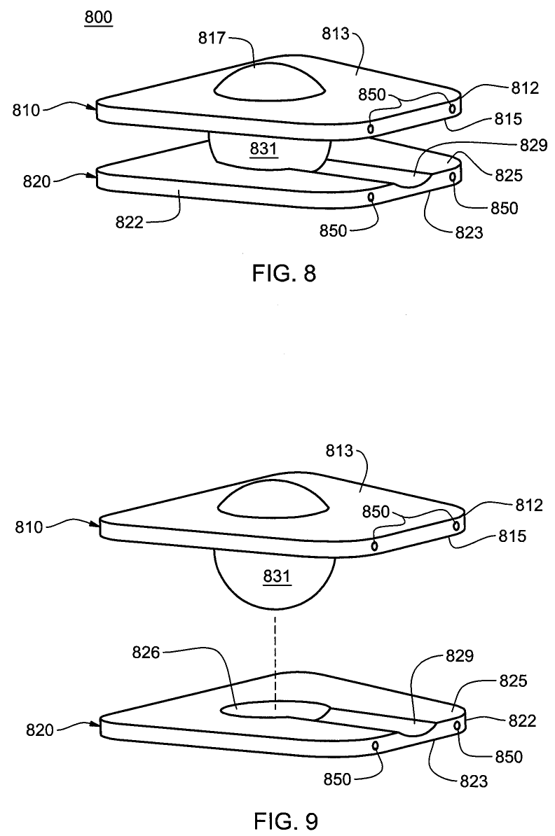

FIGS. 8- 11 depict another embodiment of an intervertebral prosthetic device 800, in accordance with an aspect of the present invention. This anterior approach embodiment is a two-component embodiment, and unless otherwise specified below, the structure, materials, configuration and function of the intervertebral prosthetic device is similar to that described above in connection with intervertebral prosthetic device 200 of FIGS. 2-5.

In the embodiment of FIGS. 8-11, two-components 810, 820 provide a ball-and-socket type joint that permits relative pivotal and rotational movement between the articular components thereof, which correspondingly permits relative pivotal and rotational movement between the superior and inferior vertebrae when positioned in the intervertebral disc space as described herein. Component 810 is configured to engage a first vertebral body and includes a first vertebral support plate 812 having a first convex protrusion 817 extending from a first surface 813 thereof. First convex protrusion 817 is placed somewhat posterior the intervertebral prosthetic device to align with the nuclear recess in a first vertebral body of the two adjacent vertebral bodies. Component 810 further includes a convex articular protrusion 831 extending from a second surface 815 of vertebral support plate 812.

Component 820 is configured similarly to component 220 of FIG. 2. Specifically, component 820 includes a second vertebral support plate 822 having a first surface 823 including a second convex protrusion 827 extending therefrom. Second convex protrusion 827 is disposed to reside within a nuclear recess of a second vertebral body when the intervertebral prosthetic device is implanted between two adjacent vertebral bodies. A concave articular recess 826 in second surface 825 of vertebral support plate 822 is configured and disposed to receive the convex articular protrusion 831 extending from second surface 815 of first vertebral support plate 812. The convex articular protrusion 831 and concave articular recess 826 function as an articulation member providing articulating motion for the intervertebral prosthetic device. More particularly, the convex and concave articular surfaces 831, 826 are positioned in abutment to allow pivotal and rotational movement therebetween. The articular recess or socket is shaped and configured to closely correspond to the shape and configuration of the convex articular protrusion. In one example, the concave articular recess and convex articular protrusion have a same radius. However, it should be understood that the radius of the convex articular protrusion may be sized somewhat smaller than the radius of curvature of the socket to facilitate articulation therebetween.

In the embodiment of FIGS. 8-11, first convex protrusion 817 from first surface 813 of first vertebral support plate 812 is a first spherical segment having a radius R from a center point 835 (see FIG. 11) and second convex protrusion 827 is a second spherical segment having radius R from center point 835 such that when the intervertebral prosthetic device 800 is implanted between adjacent vertebral bodies, the first and second spherical segments have a common center of rotation. Further, in this embodiment, the convex articular protrusion 831 extending from second surface 815 of vertebral support plate 812 is a spherical segment, and concave articular recess 826 in second surface 825 of vertebral support plate 822 is a spherical recess segment, configured to receive spherical segment 831. First spherical segment 817, articular spherical segment 831, spherical recess segment 826 and second spherical segment 827 are aligned along a central axis 840 and share a common center of rotation 835. That is, spherical segments 817, 827 are portions of a larger sphere encompassing sphere segment 831, and sharing a common center. When inserted within an intervertebral disc space, with spherical segments 817, 827 within the nuclear recesses of the adjoining vertebral bodies, central axis 840 aligns with the natural axis of rotation of the adjacent vertebrae. This facilitates maintenance or restoring of motion substantially similar to the normal bio-mechanical motion provided by a natural intervertebral disc.

As with the embodiment of FIGS. 2-5, the vertebral support surfaces of components 810, 820 are configured as bearing surfaces sized to engage the apophyseal ring of the respective vertebral bodies when the intervertebral prosthetic device is implanted in an intervertebral disc space. In one embodiment, these bearing surfaces 813, 823 are at least partially coated with a bone-growth substance, as described above.

Those skilled in the art should note that in the two-component device configuration of FIGS. 8-11, component 810 is a unitary structure, either having been assembled from discrete components or formed as a single component, for example, via a molding operation. Because convex articular surface 831 is integrated with vertebral support plate 812, only a single trough 829 is employed. Trough 829 extends from an anterior edge of second vertebral support plate 822 to convex articular recess 826 to facilitate placement of component 810 within an intervertebral disc space subsequent to placement of component 820, again, assuming a direct or oblique anterior insertion trajectory. As with the embodiment of FIGS. 2-5, trough 829 could alternatively be disposed to extend laterally or posteriorally from the convex articular recess to an edge of the device, for example, for a lateral or posterior insertion approach, respectively.

Further, in this embodiment, four insertion notches 850 are again disposed in the anterior edge of the device to facilitate manipulation and placement of the components in proper engaging position with the respective vertebral bodies.

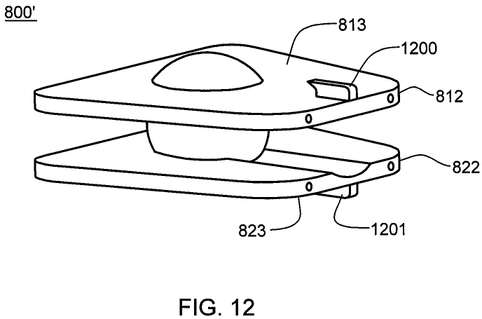

FIG. 12 depicts an enhanced embodiment of an intervertebral prosthetic device 800′, which is substantially identical to intervertebral prosthetic device 800 of FIGS. 8-11, except for the addition of gripping elements 1200, 1201 for gripping vertebral bone when the intervertebral prosthetic device 800′ is implanted between adjacent vertebral bodies. Gripping elements 1200, 1201 may be any gripping structure such as described above in connection with gripping elements 700, 701 of the intervertebral prosthetic device 200′ embodiment of FIG. 7. In this example, gripping elements 1200,1201 protrude from vertebral support surface 813 of vertebral support plate 812 and vertebral support surface 823 of vertebral support plate 822.

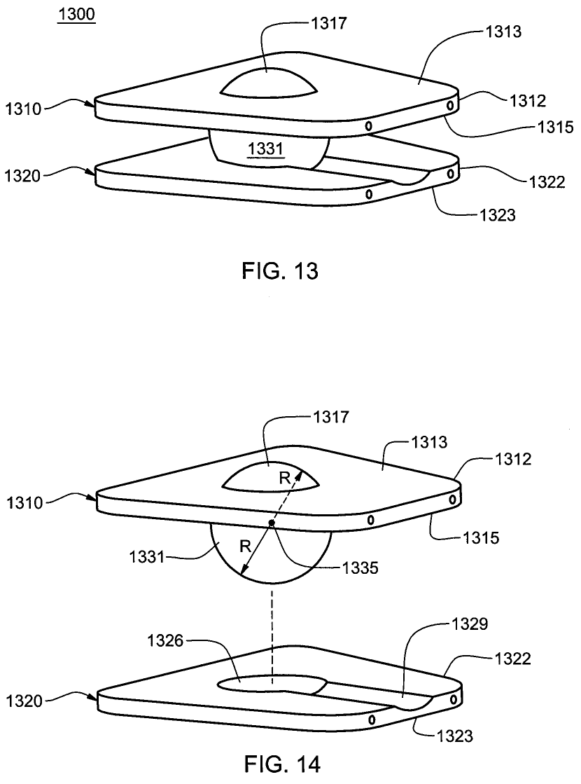

FIGS. 13 & 14 depict a further variation on the two-component intervertebral prosthetic device of FIGS. 8-11. This intervertebral prosthetic device 1300 is substantially identical to intervertebral prosthetic device 800, with the exception that component 1310 is shown to include a nuclear recess engaging spherical segment 1317 protruding from vertebral support surface 1313 of vertebral support plate 1312 which has a radius R from a center point 1335 that coincides with the radius R of articulating spherical segment 1331 protruding from a second surface 1315 of vertebral support plate 1312. Thus, in this example, the articulating spherical segment 1331 and the nuclear recess engaging spherical segment 1317 have a common center of rotation, while the other nuclear recess engaging spherical segment (not shown) protruding from vertebral support surface 1323 of vertebral support plate 1322 would have a center of rotation slightly offset therefrom, but along the same axis of rotation, which (as noted above) substantially aligns with the natural axis of rotation of the spine when the spherical segments protruding from the vertebral support surfaces are disposed within the nuclear recesses of the adjacent vertebral bodies.

From the above discussion, those skilled in the art will observe that various intervertebral prosthetic devices (or articulating joints) are described herein which employ an axis of rotation which aligns with the natural axis of rotation of the adjacent vertebrae, and in most embodiments, which share a center of rotation. The result is the provision of prosthetic devices which establish articulating motion substantially similar to the normal bio-mechanical motion provided by a natural intervertebral disc.

By way of further example, a surgical method employing a three-component intervertebral prosthetic device such as described above would include: creating a window in one of an anterior, lateral or posterior approach to an intervertebral space between adjacent first and second vertebral bodies. The diseased or degenerated natural intervertebral disc is then, or has previously been, removed via a discectomy or similar surgical procedure. The motion-preserving prosthetic device is then inserted through the window into the intervertebral space using, for example, a direct anterior insertion trajectory or an oblique anterior insertion trajectory. When inserted, the prosthetic device is positioned so that the first spherical segment protruding from the first vertebral support plate resides within a nuclear recess in one vertebral body, while the second spherical segment protruding from the second vertebral support plate resides within the nuclear recess in the other vertebral body. This positions the center of rotation of the intervertebral prosthetic device along the natural axis of rotation of the adjacent vertebrae.

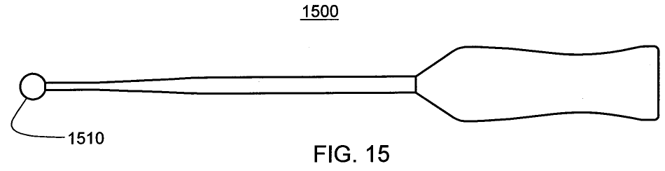

The surgical method can further include sizing the intervertebral space between the nuclear recesses in the intervertebral disc space employing one or more trial instruments. One embodiment of a trial instrument 1500 is depicted in FIG. 15, wherein the instrument includes a sphere 1510 of known size. Various trial instruments, each containing a sphere of a different size, can be employed. The sphere which provides the closest fit within the intervertebral space between the nuclear recesses is selected. A plurality of motion-preserving prosthetic devices can be provided, each sized slightly differently to accommodate different intervertebral spacing between the nuclear recesses of different patients. By way of example, the overall height of the intervertebral prosthetic device may vary between 7 mm-16 mm peak-to-peak for a lumbar prosthetic device, and between 3 mm-6 mm for a cervical prosthetic device.

Once the appropriately sized intervertebral prosthetic device is selected, the device may be inserted either assembled or by component parts. For example, in the three-component configuration of FIGS. 2-5, the vertebral support plates may first be positioned against the respective vertebral bodies, with the spherical segments thereof positioned within the nuclear recesses in the vertebral bodies. If a gripping embodiment is employed, then impaction may be used to seat the one or more gripping elements within the vertebral bone. Placement of the vertebral support plates in operative position within the intervertebral disc space is facilitated via an instrument (not shown) engaging the instrument-receiving notches in the anterior edge of the vertebral support plates. Once positioned, the articular ball may then be inserted via the troughs provided in the vertebral support plates into an operative position within the articulating recesses provided in the plates.

As another example, in the two-component approach of FIGS. 8-11, the component with the articular recess may first be inserted in operative position against one of the vertebral bodies, with the convex protrusion thereof seated within the nuclear recess of the vertebral body. Thereafter, the second component is inserted into the intervertebral disc space by disposing the convex articular protrusion within the trough of the previously-inserted vertebral support plate and sliding the second component into position, with the convex protrusion extending from the vertebral support surface thereof properly positioned within the nuclear recess of the other vertebral body.

Although certain preferred embodiments have been depicted and described in detail herein, it will be apparent to those skilled in the relevant art that various modifications, additions and substitutions can be made without departing from its essence and therefore these are to be considered to be within the scope of the following claims. For example, although the devices and methods of the present invention are particularly applicable to the lumbar region of the spine, it should nevertheless be understood that the present invention is also applicable to other portions of the spine, including the cervical or thoracic regions of the spine.

What is claimed is:

1. An intervertebral prosthetic device comprising:
a first component configured to engage a first vertebral body, the first component comprising a first vertebral support plate having a first spherical segment protruding from a first surface thereof, the first spherical segment being disposed to reside within a nuclear recess in the first vertebral body when the intervertebral prosthetic device is implanted between the first vertebral body and a second vertebral body;
a second component configured to engage the second vertebral body, the second component comprising a second vertebral support plate having a second spherical segment protruding from a first surface thereof disposed to reside within a nuclear recess in the second vertebral body when the intervertebral prosthetic device is implanted between the first vertebral body and the second vertebral body;
an articulation member interfacing the first vertebral support plate and the second vertebral support plate; and
wherein the first spherical segment protruding from the first vertebral support plate and the second spherical segment protruding from the second vertebral support plate have a common center of rotation.

2. The intervertebral prosthetic device of claim 1, wherein the articulation member comprises an articular ball, and a first concave articular surface region in a second surface of the first vertebral support plate, and a second concave articular surface region in a second surface of the second vertebral support plate, the first and second concave articular surface regions being sized to receive a portion of the articular ball, wherein the first spherical segment protruding from the first vertebral support plate, the second spherical segment protruding from the second vertebral support plate, and the articular ball share a common center of rotation.

3. The intervertebral prosthetic device of claim 2, wherein the first surface of the first vertebral support plate is a first bearing surface configured to engage the apophyseal ring of the first vertebral body when the intervertebral prosthetic device is implanted between the first vertebral body and the second vertebral body, and the first surface of the second vertebral support plate is a second bearing surface configured to engage the apophyseal ring of the second vertebral body when the intervertebral prosthetic device is implanted between the first vertebral body and the second vertebral body.

4. The intervertebral prosthetic device of claim 3, wherein the first bearing surface and the second bearing surface are each at least partially coated with a bone-growth promoting substance.

5. The intervertebral prosthetic device of claim 3, wherein the first and second vertebral support plates further comprise at least one gripping element configured to grip vertebral bone when the intervertebral prosthetic device is implanted between the first vertebral body and the second vertebral body.

6. The intervertebral prosthetic device of claim 2, wherein the first component and the second component are identical components.

7. The intervertebral prosthetic device of claim 2, wherein the second surfaces of the first and second vertebral support plates include aligned troughs extending from an edge of the intervertebral prosthetic device to the first and second concave articular surface regions, the troughs facilitating insertion of the articular ball in operative position between the first and second concave articular surface regions after insertion of the first vertebral support plate in engagement with the first vertebral body and the second vertebral support plate in engagement with the second vertebral body.

8. The intervertebral prosthetic device of claim 7, wherein the troughs in the second surfaces of the first and second vertebral support plates respectively extend from an anterior edge of the intervertebral prosthetic device to one of the first and second concave articular surface regions.

9. The intervertebral prosthetic device of claim 2, wherein the first and second components and the articular ball are formed of a biocompatible material.

10. The intervertebral prosthetic device of claim 1, wherein the first and second components each comprise at least one notch formed anteriorly therein for receiving a surgical instrument to facilitate placement of the first and second components in engagement with a respective one of the first and second vertebral bodies.

11. The intervertebral prosthetic device of claim 1, wherein the articulation member comprises a first articular surface region of the first component and a second articular surface region of the second component, the first articular surface region of the first component comprising a convex articular surface region and the second articular surface region of the second component comprising a concave articular surface region, wherein the convex articular surface region cooperates with the concave articular surface region to provide articulating motion.

12. The intervertebral prosthetic device of claim 11, wherein the convex articular surface region comprises a portion of a second surface of the first vertebral support plate, and the concave articular surface region comprises a portion of a second surface of the second vertebral support plate.

13. The intervertebral prosthetic device of claim 12, wherein the convex articular surface region comprises an articular spherical segment protruding from the second surface of the first vertebral support plate and the concave articular surface region comprises an articular spherical recess segment in the second surface of the second vertebral support plate, wherein the first and articular spherical segments protruding from the first and second surfaces of the first vertebral support plate, the articular spherical recess segment in the second surface of the second vertebral support plate and the second spherical segment protruding from the first surface of the second vertebral support plate are aligned along a central axis.

14. The intervertebral prosthetic device of claim 12, wherein the articular spherical segment protruding from the second surface of the first vertebral support plate has the common center of rotation of the first spherical segment protruding from the first surface of the first vertebral support plate and the second spherical segment protruding from the first surface of the second vertebral support plate.

15. The intervertebral prosthetic device of claim 11, wherein the first component is a unitary structure.

16. The intervertebral prosthetic device of claim 11, wherein the first surface of the first vertebral support plate is a first bearing surface configured to engage the apophyseal ring of the first vertebral body when the intervertebral prosthetic device is implanted between the first vertebral body and the second vertebral body, and the first surface of the second vertebral support plate is a second bearing surface configured to engage the apophyseal ring of the second vertebral body when the intervertebral prosthetic device is implanted between the first vertebral body and the second vertebral body.

17. The intervertebral prosthetic device of claim 16, wherein the first bearing surface and the second bearing surface are each at least partially coated with a bone-growth promoting substance.

18. The intervertebral prosthetic device of claim 11, wherein the first and second vertebral support plates further comprise at least one gripping element configured to grip vertebral bone when the intervertebral prosthetic device is implanted between the first vertebral body and the second vertebral body.

19. The intervertebral prosthetic device of claim 12, wherein the second surface of the second vertebral support plate includes at least one trough extending from an edge of the second vertebral support plate to the concave articular surface region thereof, the at least one trough facilitating insertion of one of the first component and the second component into the intervertebral disc space between the first vertebral body and the second vertebral body, with the other of the first component and the second component being in the intervertebral disc space in engagement with a respective one of the first vertebral body and the second vertebral body.

20. The intervertebral prosthetic device of claim 19, wherein the at least one trough in the second surface of the second vertebral support plate comprises a trough extending from an anterior edge of the intervertebral prosthetic device to the concave articular surface region thereof.

21. The intervertebral prosthetic device of claim 11, wherein the first and second components are formed of a biocompatible material.

22. An intervertebral prosthetic device comprising:
a first component configured to engage a first vertebral body, the first component comprising a first vertebral support plate having a first convex protrusion extending from a first surface thereof, and a convex articular protrusion extending from a second surface thereof, the first convex protrusion being disposed to reside within a nuclear recess in the first vertebral body when the intervertebral prosthetic device is implanted between the first vertebral body and a second vertebral body; and
a second component configured to engage the second vertebral body, the second component comprising a second vertebral support plate having a second convex protrusion extending from a first surface thereof disposed to reside within a nuclear recess in the second vertebral body when the intervertebral prosthetic device is implanted between the first vertebral body and the second vertebral body, and a concave articular recess in a second surface thereof configured and disposed to at least partially receive the convex articular protrusion extending from the second surface of the first vertebral support plate when the intervertebral prosthetic device is implanted between the first vertebral body and the second vertebral body, wherein the convex articular protrusion and the concave articular recess function as an articulation member providing articulating motion for the intervertebral prosthetic device.

23. The intervertebral prosthetic device of claim 22, wherein the first convex protrusion from the first surface of the first vertebral support plate is a first spherical segment having a radius R and the second convex protrusion from the first surface of the second vertebral support plate is a second spherical segment having radius R, wherein when the intervertebral prosthetic device is implanted between the first vertebral body and the second vertebral body, the first and second spherical segments having radius R share a common center of rotation.

24. The intervertebral prosthetic device of claim 23, wherein the convex articular protrusion is an articular spherical segment protruding from the second surface of the first vertebral support plate and the concave articular recess is an articular spherical recess segment in the second surface of the second vertebral support plate, wherein the first spherical segment protruding from the first surface of the first vertebral support plate, the articular spherical segment protruding from the second surface of the first vertebral support plate, the articular spherical recess segment in the second surface of the second vertebral support plate and the second spherical segment protruding from the first surface of the second vertebral support plate are aligned along a central axis when the intervertebral prosthetic device is implanted between the first vertebral body and the second vertebral body.

25. The intervertebral prosthetic device of claim 24, wherein the articular spherical segment protruding from the second surface of the first vertebral support plate shares the common center of rotation with the first and second spherical segments protruding from the first and second vertebral support plates, respectively.

26. The intervertebral prosthetic device of claim 22, wherein the first surface of the first vertebral support plate is a first bearing surface configured to engage the apophyseal ring of the first vertebral body when the intervertebral prosthetic device is implanted between the first vertebral body and the second vertebral body, and the first surface of the second vertebral support plate is a second bearing surface configured to engage the apophyseal ring of the second vertebral body when the intervertebral prosthetic device is implanted between the first vertebral body and the second vertebral body.

27. The intervertebral prosthetic device of claim 26, wherein the first bearing surface and the second bearing surface are each at least partially coated with a bone-growth promoting substance.

28. The intervertebral prosthetic device of claim 26, wherein the first support plate and the second support plate further comprise at least one gripping element configured to grip vertebral bone when the intervertebral prosthetic device is implanted between the first vertebral body and the second vertebral body.

29. The intervertebral prosthetic device of claim 22, wherein the second surface of the second vertebral support plate further comprises at least one trough extending from an edge of the intervertebral prosthetic device to the concave articular recess, the at least one trough being sized to facilitate insertion of one of the first component and the second component into the intervertebral space between the first and second vertebral bodies with the other of the first component and the second component in engagement with one of the first vertebral body and the second vertebral body.

30. The intervertebral prosthetic device of claim 22, wherein the first convex protrusion from the first vertebral support plate is a spherical segment protruding from the first surface of the first vertebral support plate, and has a radius R from a center point, and the convex articular protrusion extending from the second surface of the first vertebral support plate is an articular spherical segment having a same radius R from the center point.

31. A surgical method comprising:
creating a window in an anterior, lateral or posterior approach to an intervertebral disc space between adjacent first and second vertebral bodies;
obtaining a motion-preserving prosthetic device comprising first and second components for implant, the first component being configured to engage the first vertebral body, and comprising a first vertebral support plate having a first spherical segment protruding from a first surface thereof, the first spherical segment being disposed to reside within a nuclear recess of the first vertebral body when the prosthetic device is implanted between the first and second vertebral bodies, and the second component being configured to engage the second vertebral body, the second component comprising a second vertebral support plate and having a second spherical segment protruding from a surface thereof disposed to reside within a nuclear recess in the second vertebral body when the prosthetic device is implanted between the adjacent first and second vertebral bodies, and an articulation member interfacing the first vertebral support plate and the second vertebral support plate, wherein the first spherical segment protruding from the first vertebral support plate and the second spherical segment protruding from the second vertebral support plate share a common center of rotation; and
inserting the motion-preserving prosthetic device through the window and into the intervertebral space with the first spherical segment protruding from the first vertebral support plate residing within the nuclear recess in the first vertebral body, and the second spherical segment protruding from the second vertebral support plate residing within the nuclear recess in the second vertebral body.

32. The method of claim 31, further comprising preparing the intervertebral space to receive the motion-preserving prosthetic device.

33. The method of claim 31, further comprising sizing the intervertebral space between the nuclear recess in the first vertebral body and the nuclear recess in the second vertebral body employing at least one trial instrument, and responsive to said sizing, selecting from a plurality of sets of first and second components comprising first and second spherical segments of various radii, a particular set of first and second components having first and second spherical segments with a radius chosen to reside within the sized intervertebral space between nuclear recesses of the first and second vertebral bodies.

34. The method of claim 31, wherein the inserting comprises separately inserting the components of the motion-preserving prosthetic device into the intervertebral space between the first and second vertebral bodies.

35. The method of claim 31, wherein the articulation member comprises an articular ball, and a first concave articular surface region in a second surface of the first vertebral support plate, and a second concave articular surface region in a second surface of the second vertebral support plate, the first and second concave articular surface regions being sized to at least partially receive the articular ball, wherein the first spherical segment protruding from the first vertebral support plate, the second spherical segment protruding from the second vertebral support plate, and the articular ball share a common center of rotation, and wherein troughs are provided in the second surfaces of the first and second vertebral support plates extending from an edge of the intervertebral prosthetic device to the first and second concave articular surface regions, respectively, and wherein the inserting comprises implanting the first and second vertebral support plates in engagement with the first and second vertebral bodies, and thereafter, implanting the articular ball between the first vertebral support plate and the second vertebral support plate employing the troughs in the second surfaces of the first and second vertebral support plates.

36. The method of claim 31, wherein the creating comprises creating an anterior approach to the intervertebral disc space, the anterior approach comprising one of a direct anterior insertion trajectory or an oblique anterior insertion trajectory.

37. A surgical method comprising:
creating a window in one of an anterior, lateral or posterior approach to an intervertebral disc space between adjacent first and second vertebral bodies;
obtaining a motion-preserving prosthetic device comprising first and second components for implant, the first component being configured to engage the first vertebral body, and comprising a first vertebral support plate having a first convex protrusion extending from a first surface thereof, and a convex articular protrusion extending from a second surface thereof, the first convex protrusion being disposed to reside within a nuclear recess in the first vertebral body when the intervertebral prosthetic device is implanted between the first and second vertebral bodies, and the second component being configured to engage the second vertebral body, the second component comprising a second vertebral support plate having a second convex protrusion extending from a first surface thereof and disposed to reside within a nuclear recess in the second vertebral body when the prosthetic device is implanted between the first and second vertebral bodies, and a concave articular recess in a second surface thereof, disposed to receive the convex articular protrusion extending from the second surface of the first vertebral support plate when the prosthetic device is implanted between the first and second vertebral bodies, wherein the convex articular protrusion and the concave articular recess function as an articulation member providing articulating motion for the prosthetic device; and
inserting the motion-preserving prosthetic device through the window and into the intervertebral space with the first convex protrusion extending from the first vertebral support plate residing within the nuclear recess in the first vertebral body, and the second convex protrusion extending from the second vertebral support plate residing within the nuclear recess in the second vertebral body.

38. The method of claim 37, wherein the first convex protrusion from the first vertebral support plate is a first spherical segment having a radius R from a center point and the second convex protrusion from the second vertebral support plate is a second spherical segment having radius R from the center point, wherein the first and second spherical segments of radius R have a common center of rotation when the motion-preserving prosthetic device is inserted into the intervertebral space between the first and second vertebral bodies.

39. The method of claim 38, wherein the second surface of the second vertebral support plate is further configured with a trough extending from an anterior edge thereof to the concave articular recess, and wherein the inserting comprises implanting the second component in engagement with the second vertebral body, and thereafter, implanting the first component in engagement with the first vertebral body, wherein the trough facilitates insertion of the first component into the intervertebral space by accommodating the convex articular protrusion extending from the second surface of the first vertebral support plate during the insertion process.

40. The method of claim 37, wherein the creating comprises creating an anterior approach to the intervertebral disc space, the anterior approach comprising one of a direct anterior insertion trajectory or an oblique anterior insertion trajectory.
